(19) United States
(12) Patent Application Publication
(10) Pub. No.: US 2004/0265364 A1
(43) Pub. Date: Dec. 30, 2004

(54) Neuropathy cream

(76) Inventors: Binnur Ozturk, Huntington, WV (US); Ahmet H. Ozturk, Huntington, WV (US)

Correspondence Address: JENNIFER MEREDITH, 315 PARK AVENUE SOUTH, FLOOR 19, NEW YORK, NY, 10010, US

(21) Appl. No.: 10/603,311

(22) Filed: Jun. 25, 2003

(51) Int. Cl. A61K 9/70; A61K 31/195

(52) U.S. Cl. 424/449; 514/561

(57) ABSTRACT

The present invention provides compositions for transdermal pain relief. According to one embodiment, there is between 2% and 4% by weight amitriptyline, between 0.2% and 0.5% by weight clonidine, between 5% and 20% by weight ketamine, between 2% and 6% by weight ketoprofen and optionally between 5% and 20% by weight ketamine.

### BACKGROUND OF THE INVENTION

The present invention relates to methods for treating or preventing pain via topical formulations that induce a local-anesthetic effect when applied to intact skin.

Pain results from the noxious stimulation of nerve endings. Nociceptive pain is caused by noxious stimulation of nociceptors (e.g., a needle stick or skin pinch), which then transmit impulses over intact neural pathways to the spinal neurons and then to the brain. Neuropathic pain is caused by damage to neural structures, such as damage to peripheral nerve endings or nociceptors, which become extremely sensitive to stimulation and can generate impulses in the absence of stimulation (e.g., herpes zoster pain after the rash has healed). Generally, such damage can be caused by a variety of means including trauma, diseases such as diabetes, herpes zoster and late-stage cancer, chemotherapy, or by a chemical injury. Peripheral nerve damage can lead to pathological states where there is a reduction in pain threshold (i.e., allodynia), an increased response to noxious stimuli (hyperalgesia), or an increased response duration (persistent pain).

In the past, patients were generally treated by administration of analgesics to relieve pain. A vast majority of such patients receive doses of these agents orally. Unfortunately, in some situations, oral administration of such agents has been associated with a variety of side effects, such as liver damage, kidney damage, gastrointestinal side effects, addiction, sedation, and/or weight gain which cannot be tolerated well by the patient. In other cases, malabsorption of oral preparations have resulted in subtherapeutic plasma levels. In other cases, the agents have relatively short plasma half-lives, necessitating inconveniently frequent dosing. In general, oral delivery involves a time delay as the analgesic is absorbed via the digestive system before entering the bloodstream. A number of agents which have traditionally been administered orally or by injection have been inappropriate or suboptimal for some patients when so-administered. There are a number of medications which, in at least some patients, are not tolerated well when orally administered (e.g. which cause undesirable gastrointestinal or other side effects) and/or which provide undesirably high or low concentrations or delayed concentrations in a target tissue.

As an alternative to oral preparations, pain can be treated locally by topically administering a local anesthetic directly to the painful area to block the nociceptive mechanistic pathway. Local anesthetics prevent the generation and conduction of nociceptive nerve impulses. Thus, for example, a local anesthetic can be injected intradermally (non-systemic injection within the skin) or topically applied at the pain area. Advantages of topical local-anesthetic administration over systemic administration of pain relievers include decrease or preclusion of side effects, improved patient compliance, and reversible action (i.e., the action can be reversed by removing the anesthetic from the application site). TRANSDERMAL AND TOPICAL DRUG DELIVERY SYSTEMS 33-112 (Tapash K. Ghosh et al. eds., 1997).

A variety of drug classes have local-anesthetic properties and can be administered in topical formulations. Traditional local anesthetics or sodium-channel blockers, such as lidocaine prevent the generation and conduction of nerve impulses by decreasing or preventing the large transient increase in the permeability of excitable membranes to Na⁺. Other agents with local-anesthetic properties include analgesics, such as non-steroidal anti-inflammatories (“NSAIDs”). N-methyl-D-aspartate (“NMDA”) receptor antagonists, such as ketamine have local-aesthetic properties and topical administration is as an effective neuropathic pain treatment. See, for example, U.S. Pat. No. 5,817,699 (issued Oct. 6, 1998). In another example, topical administration of antidepressant medications, such as amitriptyline, has been reported effective for neuropathic pain treatment. See, for example, U.S. Pat. No. 6,211,171 (issued Apr. 3, 2001); J. Sawynok et al., 82 PAIN 149 (1999). In addition, topical administration of a combination of a tricyclic antidepressant and an NMDA-receptor antagonist is reported to have excellent local-anesthetic properties when topically applied and is useful for treatment of neuropathic pain, U.S. Pat. No. 6,197,830 (issued Mar. 6, 2001).

But even though topical local-anesthetic administration to intact skin is routinely used to treat minor indications, it has not found significant use for treating more severe nociceptive and neuropathic pain because it is difficult to get significant concentrations through the skin barrier. Because of the skin's drug-permeation resistance, as little as about 1 percent and usually no more than about 15 percent of a drug in a topical formulation is bioavailable (TRANSDERMAL AND TOPICAL DRUG DELIVERY SYSTEMS 7 (Tapash K. Ghosh et al. eds., 1997)). Another problem with topical administration of pain relievers is stability of the composition. Local-anesthetics emulsion compositions are inherently unstable, and phase separation can occur during shipment and storage. Furthermore, many topical local-anesthetic compositions suffer from oxidative instability. Lecithin compositions are routinely used as bases for topical local-aesthetic compositions, but are highly oxidatively unstable (AM. PHARM. ASSOC., HANDBOOK OF PHARMACEUTICAL EXCIPIENTS 292-294, 292 (Arthur H. Kibbe ed., 3rd ed. 2000)). For example, U.S. Pat. No. 6,197,830 (issued Mar. 6, 2001) describes a lecithin-based composition for topically administering a combination of an NMDA-receptor antagonist and a tricyclic antidepressant and U.S. Pat. No. 5,817,699 (issued Oct. 6, 1998) and U.S. Pat. No. 6,017,961 (issued Jan. 25, 2000) describe topical administration of ketamine in pluronic lecithin organogel.

While topical local-anesthetic administration has advantages over systemic administration of pain relievers, they suffer from instability and poor skin-penetration properties, which limit their use to less severe pain. What are needed are stable, effective topical local-anesthetic compositions with good skin-penetration properties the avoid or reduce undesired effects such as liver damage or gastrointestinal side effects.

### SUMMARY OF THE INVENTION

The present invention provides a transdermal composition for the treatment of pain in a subject, particularly a human subject.

According to a preferred embodiment, a transdermal composition for the relief of pain in a subject is disclosed comprising: amitriptyiline, clonidine, ketamine and an anti-inflammatory in a base. The amitriptyline may be between 2% and 4% by weight, preferably 4%, the clonidine between 0.2% and 0.5% by weight, preferably 0.5%. The composition may also have gabapentin, which may be between 5% and 15% by weight, preferably 5%. The ketamine may be between 5% and 20% by weight, preferably 15%. The anti-inflammatory may be ketoprofen between 2% and 6% by weight, preferably 5%.

A transdermal composition for the relief of pain in a subject comprising: amitriptyiline, clonidine, gabapentin and ketamine in a base. The composition may also comprise 5% to 10% by weight lidocaine. The amitriptyline may be 2% to 4% by weight, clonidine may be 0.2% to 0.5% by weight, gabapentin may be 6% to 12% by weight, the ketamine may be 1% to 20% by weight.

According to another embodiment, a transdermal composition for the relief of pain in a subject is disclosed comprising: amitriptyiline, cyclobenzaprine, dexamethazone, gabapentin and an anti-inflammatory in a base. The amitriptyiline may be between 2% and 4% by weight, the cyclobenzaprine may be between 2% and 4% by weight, the dexamethazone may be between 0.2% and 0.5% by weight, and the gabapentin may be between 6% and 12% by weight. The anti-inflammatory may be ibuprofen being between 18% and 22% by weight or ketoprofen being between 2% and 4% by weight.

Another embodiment provides a transdermal composition for the relief of pain in a subject comprising: amitriptyline, cyclobenzaprine, dexamethazone, gabapentin, an anti-inflammatory and a local anesthetic in a base. The amitriptyline may be between 2% and 4% by weight, the cyclobenzaprine may between 1% and 3% by weight, preferably 2%, the dexamethazone may be between 0.3% and 0.5% by weight, preferably 0.4%. The gabapentin may be between 6% and 12% by weight. The anti-inflammatory may be between 15% by weight and 22% by weight ibuprofen, preferably 20% by weight. The anti-inflammatory may also be ketoprofen between 2% by weight and 4% by weight. The local anesthetic may be between 5% by weight and 10% by weight lidocaine. The local anesthetic may also be between 5% by weight and 10% by weight EMLA, preferably 7% by weight.

According to yet another embodiment, a transdermal composition for the relief of pain in a subject comprising: analgesic, an anti-epileptic compound, cyclobenzaprine, gabapentin, ketamine and an local anesthetic in a base. The analgesic may be between 3% and 6% by weight aspirin, preferably 5% by weight. The anti-epileptic may be between 2% and 4% by weight carbamazepine. The gabapentin is preferably between 6% and 12% by weight, the ketamine is between 13% and 16% by weight, preferably 15%. The local anesthetic may be between 5% and 10% by weight lidocaine.

According to yet another embodiment, a transdermal composition for the relief of pain in a subject is disclosed comprising: clonidine, gabapentin, ketamine, anti-inflammatory and an local anesthetic in a base. The clonidine may be between 0.2% and 0.5% by weight, the gabapentin may be between 6% and 12% by weight, the ketamine may be between 13% and 16% by weight. The anti-inflammatory may be 2% to 4% by weight ketoprofen. The local anesthetic may be 5% to 10% by weight lidocaine or 6% to 8% by weight EMLA, preferably 7% by weight EMLA.

A transdermal composition for the relief of pain in a subject comprising: amitriptyline, gabapentin, ketamine, anti-inflammatory and an local anesthetic in a base. The amitriptyline may be between 2% and 4% by weight, gabapentin may be between 6% and 12% by weight, ketamine may be between 13% and 16% by weight. The anti-inflammatory may be 2% to 4% by weight ketoprofen. The local anesthetic may be 5% to 10% by weight lidocaine or 6% to 8%, preferably 7% by weight, EMLA.

According to another embodiment, a transdermal composition for the relief of pain in a subject is disclosed, comprising: 4% by weight amitriptyiline, 0.5% by weight clonidine, 15% by weight ketamine, and 5% by weight ketoprofen in a base. This composition may be further comprised of 5% by weight gabapentin.

These and other features, aspects and advantages of the present invention will become better understood with reference to the following drawings, description and claims.

### DETAILED DESCRIPTION OF THE INVENTION

The following detailed description is of the best currently contemplated modes of carrying out the invention. The description is not to be taken in a limiting sense, but is made merely for the purpose of illustrating the general principles of the invention, since the scope of the invention is best defined by the appended claims.

The present invention provides a transdermal composition suitable for the treatment of pain in a subject. This may include neuropathic pain of all origins including shingles, post-herpetic neuralgia, diabetic neuropathy, peripheral neuropathies, intercostals neuralgia, neuralgias of the trunk and extremities. Also, the present invention may be used to treat arthritis pain, osteoarthritis, rheumatoid arthritis and other arthritic conditions. It may also be used to treat sprains, strains, fibromyalgia, muscular headaches and tension type headaches.

As used herein the term “subject” includes mammals including humans, pigs, cows, mice, rats, rabbits, goats and the like. The preferred subject is a human. As used here, the term “transdermal” composition includes compositions capable of passing through the stratum corneum of a subject. The term transdermal further includes compositions capable passing through the epidermis of a subject, compositions capable of passing through the dermis of a subject, and compositions capable of passing through the hydodermis of subject. In preferred embodiments, the term transdermal includes compositions capable of passing through the skin of a subject and reaching the underlying tissues and organs.

According to one embodiment, a transdermal composition for the relief of pain in a subject is disclosed comprising: amitriptyiline, clonidine, ketamine and an anti-inflammatory in a base. The base may be any pharmaceutically acceptable carrier which is capable of transdermal delivery of the compounds contained within the composition. This may include a variety of finite and non-finite carriers including liquids, semi-liquids or solid carriers, such as bioadhesives. By way of example, this may be creams, gels, emulsions, lotions, salves, paste, plaster, ointment, spray solution, lipids, phospholipids, lecithins, fatty oils, lanolin, vasoline, paraffins, glycols, higher fatty acids and higher alcohols. Bioadhesive bases may be natural or synthetic polysaccharides. There may also be additives including binders, stabilizers, preservatives, flavorings, fiances, and pigments.

According to a preferred embodiment, the base may be pentravan gel. Pentravan gel is an emollient which softens and moisturizes the skin. Emollients may be used as lubricants to treat or prevent dry, itchy skin and minor skin irritations. The base may also be a base comprised of approximately 80% by weight pleurinic gel and approximately 20% by weight lipoil. The proper base is extremely important, as it acts as a vehicle that allows the various drug components to penetrate the skin. Traditional cream or ointment bases would not be effective. The amitriptyline may be between 2% and 4% by weight, preferably 4%. Amitriptyline is a tricyclic antidepressant with proven efficacy on neuropathic pain when administered orally. However, oral admininistration has many serious side effects, especially in the elderly population. This may include anticholinergic effects, such as tachycardia, dry mouth and severe sedation. There may be clonidine between 0.2% and 0.5% by weight, preferably 0.5%. Clonidine is an antihypertensive medicine. The composition may also have gabapentin, which may be between 5% and 15% by weight, preferably 5%. Gabapentin is an anticonvulsant medication. The ketamine may be between 5% and 20% by weight, preferably 15%. Ketamine is generally administered by IV for general anesthesia. It has NMDA inhibitor properties and is effective in controlling pain at spinal cord level. The anti-inflammatory may be ketoprofen between 2% and 6% by weight, preferably 5%.

According to one embodiment, a transdermal composition for the relief of pain in a subject comprising: amitriptyiline, clonidine, gabapentin and ketamine in a base. The base may be pentravan gel or a composition made of 80% by weight pleurinic gel and 20% by weight lipoil. The composition may also comprise 5% to 10% by weight lidocaine. There may also be EMLA. EMLA is a local anesthetic usually used to numb the skin to pain from injections. It is also sometimes used to reduce pain associated with tattooing, electrolysis, laser hair removal, etc. It is generally comprised of lidocaine and prilocaine. The amitriptyline may be 2% to 4% by weight, clonidine may be 0.2% to 0.5% by weight, gabapentin may be 6% to 12% by weight, the ketamine may be 1% to 20% by weight.

By way of example, an order for 60 grams of neuropathy cream may be prepared by obtaining 1.2 grams amitriptyline (2% by weight), 120 mg clonidine (0.2% by weight), 9 grams gabapentin (15% by weight) 3.6 grams ketamine (6% by weight), and 4.2 grams EMLA cream (7% by weight). The powders may be mixed (e.g. in an EMP Jar) and dissolved with a small amount of grain alcohol (˜90% ethanol). Pentravan Gel may be added to make 60 grams. Finally and unguator is used to mix the final product into a very fine, smooth creme.

According to another embodiment, a transdermal composition for the relief of pain in a subject is disclosed comprising: amitriptyiline, cyclobenzaprine, dexamethazone, gabapentin and an anti-inflammatory in a base. The base is may be pentravan gel or a base made of 80% by weight pleurinic gel and 20% by weight lipoil. The amitriptyiline may be between 2% and 4% by weight, the cyclobenzaprine may be between 2% and 4% by weight, the dexamethazone may be between 0.2% and 0.5% by weight, and the gabapentin may be between 6% and 12% by weight. The anti-inflammatory may be ibuprofen being between 18% and 22% by weight. The anti-inflammatory may also be ketoprofen being between 2% and 4% by weight.

Another embodiment provides a transdermal composition for the relief of pain in a subject comprising: amitriptyline, cyclobenzaprine, dexamethazone, gabapentin, an anti-inflammatory and a local anesthetic in a base. As previously, the base may be pentravan gel or a base made of 80% by weight pleurinic gel and 20% by weight lipoil. The amitriptyline may be between 2% and 4% by weight, the cyclobenzaprine may between 1% and 3% by weight, preferably 2%, the dexamethazone may be between 0.3% and 0.5% by weight, preferably 0.4%. The gabapentin may be between 6% and 12% by weight. The anti-inflammatory may be between 15% by weight and 22% by weight ibuprofen, preferably 20% by weight. The anti-inflammatory may also be ketoprofen between 2% by weight and 4% by weight. The local anesthetic may be between 5% by weight and 10% by weight lidocaine. Lidocaine is generally used in injectable form for local, regional and neuroaxial anesthesia. While Lidocaine has been used as a topical agent, it has limited benefit when used as a sole agent. The local anesthetic may also be between 5% by weight and 10% by weight EMLA, preferably 7% by weight. EMLA is a cream, and is no longer readily available. However, the term is intended to denote a cream that has 2.5% Lidocaine and 2.5% Prilocaine.

According to yet another embodiment, a transdermal composition for the relief of pain in a subject comprising: analgesic, an anti-epileptic compound, cyclobenzaprine, gabapentin, ketamine and an local anesthetic in a base. The analgesic may be between 3% and 6% by weight aspirin, preferably 5% by weight. The anti-epileptic may be between 2% and 4% by weight carbamazepine. Carbamazepine is a mood stabilizing medication, which may be sold under a variety of trade names including Tegretol. Carbamazepine is frequently used in psychiatric practice as either augmentation medications (to render antidepressants more effective) or as anti-manic medications in the treatment of bipolar mood disorder. Mood stabilizing medications are also used in neurologic practice for the treatment of seizure disorders and for the treatment of certain pain disorders. The gabapentin is preferably between 6% and 12% by weight, the ketamine is between 13% and 16% by weight, preferably 15%. The local anesthetic may be between 5% and 10% by weight lidocaine.

According to yet another embodiment, a transdermal composition for the relief of pain in a subject is disclosed comprising: clonidine, gabapentin, ketamine, anti-inflammatory and an local anesthetic in a base. The clonidine may be between 0.2% and 0.5% by weight, the gabapentin may be between 6% and 12% by weight, the ketamine may be between 13% and 16% by weight. The anti-inflammatory may be 2% to 4% by weight ketoprofen. The local anesthetic may be 5% to 10% by weight lidocaine or 6% to 8% by weight EMLA, preferably 7% by weight EMLA.

A transdermal composition for the relief of pain in a subject comprising: amitriptyline, gabapentin, ketamine, anti-inflammatory and an local anesthetic in a base. The amitriptyline may be between 2% and 4% by weight, gabapentin may be between 6% and 12% by weight, ketamine may be between 13% and 16% by weight. The anti-inflammatory may be 2% to 4% by weight ketoprofen. The local anesthetic may be 5% to 10% by weight lidocaine or 6% to 8%, preferably 7% by weight, EMLA.

According to another embodiment, a transdermal composition for the relief of pain in a subject is disclosed, comprising: 4% by weight amitriptyiline, 0.5% by weight clonidine, 15% by weight ketamine, and 5% by weight ketoprofen in a base. This composition may be further comprised of 5% by weight gabapentin.

It should be understood that the foregoing relates to preferred embodiments of the invention and that modifications may be made without departing from the spirit and scope of the invention as set forth in the following claims.

We claim:

1. A transdermal composition for the relief of pain in a subject comprising:
amitriptyiline, clonidine, ketamine and an anti-inflammatory in a base.

2. A transdermal composition as in claim 1, wherein said base is pentravan gel.

3. A transdermal composition as in claim 1, wherein said base is 80% by weight pleurinic gel and 20% by weight lipoil.

4. A transdermal composition as in claim 1, wherein said amitriptyline is between 2% and 4% by weight, preferably 4%.

5. A transdermal composition as in claim 1, wherein said clonidine is between 0.2% and 0.5% by weight, preferably 0.5%.

6. A transdermal composition as in claim 1, further comprising gabapentin.

7. A transdermal composition as in claim 6, wherein said gabapentin is between 5% and 15% by weight, preferably 5%.

8. A transdermal composition as in claim 1, wherein said ketamine is between 5% and 20% by weight, preferably 15%.

9. A transdermal composition as in claim 1, wherein said anti-inflammatory is ketoprofen between 2% and 6% by weight, preferably 5%.

10. A transdermal composition for the relief of pain in a subject comprising:
amitriptyiline, clonidine, gabapentin and ketamine in a base.

11. A transdermal composition as in claim 10, wherein said base is pentravan gel.

12. A transdermal composition as in claim 10, wherein said base is 80% by weight pleurinic gel and 20% by weight lipoil.

13. A transdermal composition as in claim 10, further comprising lidocaine.

14. A transdermal composition as in claim 10, further comprising EMLA.

15. A transdermal composition as in claim 10, wherein said amitriptyline is 2% to 4% by weight.

16. A transdermal composition as in claim 10, wherein said clonidine is 0.2% to 0.5% by weight.

17. A transdermal composition as in claim 10, wherein said gabapentin is 6% to 12% by weight.

18. A transdermal composition as in claim 10, wherein said ketamine is 1% to 20% by weight.

19. A transdermal composition for the relief of pain in a subject comprising:
amitriptyiline, cyclobenzaprine, dexamethazone, gabapentin and an anti-inflammatory in a base.

20. A transdermal composition as in claim 19, wherein said base is pentravan gel.

21. A transdermal composition as in claim 19, wherein said base is 80% by weight pleurinic gel and 20% by weight lipoil.

22. A transdermal composition as in claim 19, wherein said amitriptyiline is between 2% and 4% by weight.

23. A transdermal composition as in claim 19, wherein said cyclobenzaprine is between 2% and 4% by weight.

24. A transdermal composition as in claim 19, wherein said dexamethazone is between 0.2% and 0.5% by weight.

25. A transdermal composition as in claim 19, wherein said gabapentin is between 6% and 12% by weight.

26. A transdermal composition as in claim 19, wherein said anti-inflammatory is ibuprofen being between 18% and 22% by weight.

27. A transdermal composition as in claim 19, wherein said anti-inflamatory is ketoprofen being between 2% and 4% by weight.

28. A transdermal composition for the relief of pain in a subject comprising:
amitriptyline, cyclobenzaprine, dexamethazone, gabapentin, an anti-inflammatory and an local anesthetic in a base.

29. A transdermal composition as in claim 28, wherein said base is pentravan gel.

30. A transdermal composition as in claim 28, wherein said base is 80% by weight pleurinic gel and 20% by weight lipoil.

31. A transdermal composition as in claim 28, wherein said amitriptyline is between 2% and 4% by weight.

32. A transdermal composition as in claim 28, wherein said cyclobenzaprine is between 1% and 3% by weight, preferably 2%.

33. A transdermal composition as in claim 28, wherein said dexamethazone is between 0.3% and 0.5% by weight, preferably 0.4%.

34. A transdermal composition as in claim 28, wherein said gabapentin is between 6% and 12% by weight.

35. A transdermal composition as in claim 28, wherein said anti-inflammatory is ibuprofen between 15% by weight and 22% by weight, preferably 20% by weight.

36. A transdermal composition as in claim 28, wherein said anti-inflammatory is ketoprofen between 2% by weight and 4% by weight.

37. A transdermal composition as in claim 28, wherein said local anesthetic is lidocaine between 5% by weight and 10% by weight.

38. A transdermal composition as in claim 28, wherein said local anesthetic is EMLA between 5% by weight and 10% by weight, preferably 7% by weight.

39. A transdermal composition for the relief of pain in a subject comprising:
analgesic, an anti-epileptic compound, cyclobenzaprine, gabapentin, ketamine and an local anesthetic in a base.

40. A transdermal composition as in claim 39, wherein said base is pentravan gel.

41. A transdermal composition as in claim 39, wherein said base is 80% by weight pleurinic gel and 20% by weight lipoil.

42. A transdermal composition as in claim 39, wherein said analgesic is between 3% and 6% by weight aspirin, preferably 5% by weight.

43. A transdermal composition as in claim 28, wherein said anti-epileptic is between 2% and 4% by weight carbamazepine.

44. A transdermal composition as in claim 28, wherein said gabapentin is between 6% and 12% by weight.

45. A transdermal composition as in claim 28, wherein said ketamine is between 13% and 16% by weight, preferably 15%.

46. A transdermal composition as in claim 28, wherein said local anesthetic is between 5% and 10% by weight lidocaine.

47. A transdermal composition for the relief of pain in a subject comprising:
clonidine, gabapentin, ketamine, anti-inflammatory and an local anesthetic in a base.

48. A transdermal composition as in claim 47, wherein said base is pentravan gel.

49. A transdermal composition as in claim 47, wherein said base is 80% by weight pleurinic gel and 20% by weight lipoil.

50. A transdermal composition as in claim 47, wherein said clonidine is between 0.2% and 0.5% by weight.

51. A transdermal composition as in claim 47, wherein said gabapentin is between 6% and 12% by weight.

52. A transdermal composition as in claim 47, wherein said ketamine is between 13% and 16% by weight.

53. A transdermal composition as in claim 47, wherein said anti-inflammatory is ketoprofen, said ketoprofen being 2% to 4% by weight.

54. A transdermal composition as in claim 47, wherein said local anesthetic is 5% to 10% by weight lidocaine.

55. A transdermal composition as in claim 47, wherein said local anesthetic is 6% to 8% by weight EMLA, preferably 7% by weight EMLA.

56. A transdermal composition for the relief of pain in a subject comprising:
amitriptyline, gabapentin, ketamine, anti-inflammatory and an local anesthetic in a base.

57. A transdermal composition as in claim 56, wherein said base is pentravan gel.

58. A transdermal composition as in claim 56, wherein said base is 80% by weight pleurinic gel and 20% by weight lipoil.

59. A transdermal composition as in claim 56, wherein said amitriptyline is between 2% and 4% by weight.

60. A transdermal composition as in claim 56, wherein said gabapentin is between 6% and 12% by weight.

61. A transdermal composition as in claim 56, wherein said ketamine is between 13% and 16% by weight.

62. A transdermal composition as in claim 56, wherein said anti-inflammatory is ketoprofen, said ketoprofen being 2% to 4% by weight.

63. A transdermal composition as in claim 56, wherein said local anesthetic is 5% to 10% by weight lidocaine.

64. A transdermal composition as in claim 56, wherein said local anesthetic is 6% to 8% by weight EMLA, preferably 7% by weight EMLA.

65. A transdermal composition for the relief of pain in a subject comprising:
4% by weight amitriptyiline, 0.5% by weight clonidine, 15% by weight ketamine, and 5% by weight ketoprofen in a base.

66. A transdermal composition as in claim 65, wherein said anti-inflammatory is ibufrpofen between 15% by weight and 22% by weight, preferably 20% by weight.

67. A transdermal composition as in claim 65, further comprising 5% by weight gabapentin.
